(19) United States
(12) Patent Application Publication
(10) Pub. No.: US 2013/0045931 A1
(43) Pub. Date: Feb. 21, 2013

(54) PRODUCTION AND USE OF ANTIBACTERIAL, ANTIPROLIFERATIVE, AND ANTIPHYTOPATHOGENIC BENZANTHRACENES

(71) Applicants: Johannes Imhoff, Preetz (DE); Jutta Wiese, Eutin (DE); Heidi Zinecker, Kiel (DE); Inga Knopf-Kajahn, Gettorf (DE); Kerstin Nagel, Fleckeby (DE); Imke Schneemann, Kiel (DE)

(72) Inventors: Johannes Imhoff, Preetz (DE); Jutta Wiese, Eutin (DE); Heidi Zinecker, Kiel (DE); Inga Knopf-Kajahn, Gettorf (DE); Kerstin Nagel, Fleckeby (DE); Imke Schneemann, Kiel (DE)

(73) Assignee: Geomar Helmotz-Zentrum Für Ozeanforschung Kiel, Kiel (DE)

(21) Appl. No.: 13/575,416

(22) PCT Filed: Nov. 23, 2010
(86) PCT No.: PCT/DE2010/001375

§ 371 Date: Oct. 19, 2012

(30) Foreign Application Priority Data
Jan. 28, 2010 (DE) 10 2010 006 245.6

(51) Int. Cl. C07H 7/04; A61K 31/704; A61P 35/00; A01P 1/00; A61P 31/04; A01N 43/16; A01P 3/00; C12P 19/26; A61P 31/00

(52) U.S. Cl. 514/23; 435/84; 536/18.7

(57) ABSTRACT

The invention relates to a compound of the general structure (I), where R is a hydrogen atom (H) or an unsubstituted, monosubstituted, or polysubstituted C₁-C₂₀ alkyl, wherein the alkyl can be straight, branched, cyclic, or partially unsaturated, or an unsubstituted, monosubstituted, or polysubstituted phenyl group.

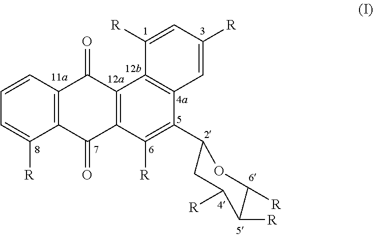

The present invention relates to novel benzanthrins, the mayamycins, which are suitable for the preparation of medicaments for the treatment of infectious diseases and tumor diseases. A further field of application of the invention is the preparation of plant protectants. The invention furthermore relates to a process for the production of these compounds by fermenting a bacterium and chromatographic purification from the bacterial extract, and to their use as medicament, cosmetic and plant protectant.

The antibiotics benzanthrin A and B were isolated from the bacterium Nocardia lurida (Rasmussen et al., 1986 Benzanthrins A and B, a new class of quinine antibiotics. Journal of Antibiotics 39: 1515-6) and are active against some Gram-positive bacteria, but not against Gram-negative bacteria (Theriault et al., 1986 Benzanthrins A and B, a new class of quinine antibiotics. Journal of Antibiotics 39: 1509-14). In addition to the antimicrobial activity, the cytotoxic activity of these substances is also known from EP0180045.

There is worldwide an increase in demand for novel active substances for the treatment of infectious diseases and tumor diseases. There is furthermore a demand for novel compounds for cosmetic products. Furthermore, there is a demand for novel substances which can be employed as plant protectants.

The present invention is therefore based on the object of providing novel benzanthrins which have antibiotic activity, antiproliferative activity or activity against the causative agents of plant diseases, and to show a route for producing them.

According to the invention, this object is achieved by the compound with the characteristics of the main claim. The dependent claims are advantageous embodiments of the invention.

In the context of a scientific program for isolating biologically active natural products from micro-organisms, the inventors have isolated the bacterium Streptomyces sp. HB202 from the sponge Halichondria panicea and studied it.

The benzanthrin mayamycin according to the invention

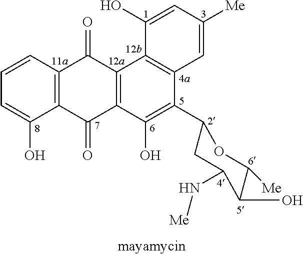 [SMILES: CNC1CC(c2c(O)c3c(c4c(O)cc(C)cc24)C(=O)c2cccc(O)c2C3=O)OC(C)C1O]

has been isolated from cultures of the bacterium Streptomyces sp. HB202 (examples 1, 2) by ethyl acetate extraction and preparative HPLC.

The mayamycin according to the invention has a structure which is similar to that of benzanthrins A and B. However, mayamycin has a hydroxyl group at position C-1, while an O-glycosidically bound amino sugar is located at that position in the case of the two benzanthrins. Furthermore, the second amino sugar in the benzanthrins is glycosylically bound at C-2, while it is glycosylically bound to C-5 in the case of mayamycin. Therefore, mayamycin is a novel compound in which only the skeleton is identical to that of the known benzanthrins. Mayamycin therefore belongs to a group of compounds, the mayamycins, with the general structure:

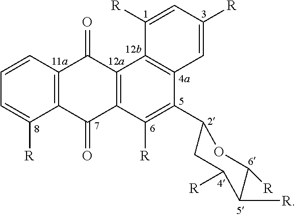

The physical data of mayamycin are shown in the appended table 1, which shows the NMR data (600 MHz, MeOD) of mayamycin.

The substances according to the invention, in the specific embodiment mayamycin, are capable of inhibiting the growth of Bacillus subtilis (DSM 347) and Staphylococcus lentus (DSM 6672). Furthermore, mayamycin exhibits a significant inhibitory effect against clinically relevant bacterial strains such as Staphylococcus aureus (ATCC 12600), Staphylococcus aureus (ATCC 33593, methicillin-resistant), Klebsiella pneumoniae (ATCC 700603, extended β-lactamase resistance) and against the biofilm formers Staphylococcus epidermidis and Pseudomonas aeruginosa (ATCC 10145) (example 3; table 2). Furthermore, mayamycin also inhibits the growth of not only the causative agent of acne vulgaris, Propionibacterium acnes (DSM 1897) (example 4; table 2), but also the growth of the phytopathogenic bacterium Xanthomonas campestris (DSM 2405), the causative agent of, inter alia, black rot in cabbage, and of the phytopathogenic fungus Phytophthora infestans, the causative agent of potato late blight (example 5; table 2).

Furthermore, the substance mayamycin according to the invention also shows different significant inhibitory effects against tumor cell lines, in particular against human liver tumor cell lines, intestinal tumor cell lines, gastric tumor cell lines, lung tumor cell lines, breast cancer cell lines, melanoma cell lines, pancreatic tumor cell lines and kidney tumor cell lines (example 6; table 3).

The culturing of Streptomyces sp. HB202 and the purification of mayamycin from cultures of the bacterium and the determination of the biological activity are described in the following examples.

### EXAMPLE 1

### Biotechnological Production of Mayamycin from Streptomyces sp. HB202

The strain Streptomyces sp. HB202 was isolated from the sponge Halichondria panicea as described by Mitova et al. (2008 Subinhibitory concentrations of antibiotics induce phenazine production in a marine Streptomyces sp. Journal of Natural Products 71: 824-7) and identified. Culturing was done for 7 days in 2 1 penicillin flasks at 28° C. The culturing and production of mayamycin may also be done in customary fermenter systems.

### EXAMPLE 2

### Isolation of Mayamycin from the Bacterial Culture

The culture solution (3 l) was harvested, homogenized for 30 seconds at 16 000 rpm using the Ultra Turrax T25 basic (IKA-Werke GmbH and Co., Staufen, Germany) and then extracted with ethyl acetate (660 ml per liter of culture solution). The ethyl acetate phase was dried, resuspended in methanol and purified further by preparative HPLC:

Separating column: Phenomenex Gemini-NX C18 110A, 100×50.00 mm

Solvent: water+0.1% formic acid (A), acetonitrile+0.1% formic acid (B)

Gradient: 0 min−10% B, 17 min−60% B, 22 min−100% B

Flow rate: 100 ml/min

Mayamycin (11.6 mg) eluted after 8.4-9.0 min.

### EXAMPLE 3

### Antibacterial Activity of Mayamycin

Mayamycin inhibited the growth of both Bacillus subtilis (DSM 347) and of Staphylococcus lentus (DSM 6672) with an 1050 value of 8 μM. The experiments were carried out as described by Lang et al. (2007 New pentaenes from sponge-derived marine fungus Penicillium rugulosum: structure determination and biosynthetic studies. Tetrahedron 63: 11844-9). The 1050 values for clinically relevant bacterial strains were 2.5 μM for Staphylococcus aureus (ATCC 12600), 1.25 μM for Staphylococcus aureus (ATCC 33593, methicillin-resistant), 2.5 μM for Klebsiella pneumoniae (ATCC 700603, extended β-lactamase resistance) and 0.31 μM and 2.5 μM, respectively, for the biofilm formers Staphylococcus epidermidis and Pseudomonas aeruginosa (ATCC 10145). The tests were carried out as described by Sahly et al. (2003 Burkholderia is highly resistant to human Beta-defensin 3. Antimicrobial Agents and Chemotherapy 47: 1739-41).

### EXAMPLE 4

### Antibacterial Activity of Mayamycin Against the Causative Agent of Acne Vulgaris

The causative agent of acne vulgaris, Propionibacterium acnes (DSM 1897), was inhibited by mayamycin with an IC50 value of 31.2 μM. The test was performed as described by Lang et al. (2007 New pentaenes from sponge-derived marine fungus Penicillium rugulosum: structure determination and biosynthetic studies. Tetrahedron 63: 11844-9).

### EXAMPLE 5

### Antibacterial and Fungicidal Activity of Mayamycin Against Causative Agents of Plant Diseases

The phytopathogenic bacterium Xanthomonas campestris (DSM 2405), inter alia the causative agent of black rot in cabbage, was inhibited by mayamycin with an IC50 value of 30 μM. The test was performed as described by Lang et al. (2007 New pentaenes from sponge-derived marine fungus Penicillium rugulosum: structure determination and biosynthetic studies. Tetrahedron 63: 11844-9). In addition, the phytopathogenic fungus Phytophthora infestans—the causative agent of potato late blight—was inhibited by mayamycin with an IC50 value of 15.2 μM. The inhibitory activity was detected in a microtiter plate. 190 μl of a spore suspension of Phytophthora infestans were seeded into each well at a concentration of 1×10⁴ spores/ml. A dilution series was prepared from a 10 mM mayamycin stock solution in dimethyl sulfoxide. 10 μl were employed per well. Incubation was performed for 48 hours at 20° C. in the dark. The absorption was measured at 600 nm. 10 μM cycloheximide were employed as the positive control.

### EXAMPLE 6

### Antiproliferative Activity of Mayamycin

To determine the proliferation and cytotoxicity in respect of the liver tumor cell line HepG2 and the intestinal tumor cell line HT29, the effect on the metabolic activity of the cells was studied by means of the CellTiter-Blue® cell viability assay as described by O'Brien et al. (2000 Investigation of the alamar blue (resazurin) fluorescent dye for the assessment of mammalian cell cytotoxicity. European Journal of Biochemistry 267: 5421-6). The antiproliferative activity on the cell lines GXF251L (gastric tumor), LXF529L (lung tumor), MAXF401NL (breast cancer), MEXF462NL (melanoma), PAXF1657L (pancreatic tumor) and RXF486L (kidney tumor) was also determined as described by Dengler et al. (1995 Development of a propidium iodide fluorescence assay for proliferation and cytotoxicity assay. Anti-Cancer Drugs 6: 522-32). The results are shown in table 3.

TABLE 1
NMR data (600 MHz, MeOD) of mayamycin
Dark brown amorphous solid.
Optical activity: [α]²⁵_{D} 0° (c 0.25, MeOH)
HRESIMS: m/z 464.17072 [M + H]⁺ (calculated for C₂₆H₂₆NO₇, 464.17038)

| Position | δ_{C}, mult. | δ_{H}(J in Hz) | COSY | HMBC | NOESY |
|---|---|---|---|---|---|
| 1 | 156.6, qC | | | | |
| 2 | 114.7, CH | 6.74, s | | 4, 3-CH₃, 12b | |
| 3 | 143.4, qC | | | | |
| 3-CH₃ | 22.6, CH₃ | 2.45, s | | 2, 3, 4 | 4 |
| 4 | 117.6, CH | 8.00, s | | 2, 3-CH₃, 4a, 5, 12b | 3-CH₃, 2′ |
| 4a | 139.9, qC | | | | |
| 5 | 126.5, qC^{b} | | | | |
| 6 | 154.4, qC^{b} | | | | |
| 6a | 119.4/138.4, qC | | | | |
| 7 | 194.2, qC | | | | |
| 7a | 116.3, qC | | | | |
| 8 | 162.9, qC | | | | |
| 9 | 124.9, CH | 7.29, dd (1.2, 8.5)^{a} | 10 | 7a, 11 | 10 |
| 10 | 138.8, CH | 7.75, dd (7.5, 8.5)^{a} | 9, 11 | 8, 11, 11a | 9, 11 |
| 11 | 120.4, CH | 7.58, dd (1.2, 7.5)^{a} | 10 | 7a, 9, 12 | 10 |
| 11a | 137.9, qC | | | | |
| 12 | 188.0, qC^{b} | | | | |
| 12a | 119.4/138.4, qC | | | | |
| 12b | 117.8, qC | | | | |
| 2′ | 72.8, CH | 5.70, dd (10.7, 2.0) | 3′a, 3′b | 4a, 5, 6 | 4, 3′b, 4′, 6′ |
| 3′a | 32.9, CH₂ | 2.35, q (12.0) | 2′, 4′ | 2′, 4′, 5′ | 4′, 4′-N—CH₃ |
| 3′b | 32.9, CH₂ | 2.27, ddd (12.9, 2.0, 4.4) | 2′, 4′ | 2′, 4′, 5′ | 2′, 4′, 4′-N—CH₃ |
| 4′ | 63.0, CH | 3.22, br, m | 3′, 5′ | | 2′, 3′a, 3′b, 4′-N—CH₃ |
| 4′-N—CH₃ | 31.4, CH₃ | 2.63, s | | 4′ | 3′a, 3′b, 4′ |
| 5′ | 74.5, CH | 3.41, t (9.5) | 4′, 6′ | 4′, 6′, 6′-CH3 | 4′, 6′, 6′-CH₃ |
| 6′ | 79.2, CH | 3.56, dq (9.5, 6.1) | 5′, 6′-CH₃ | 4′, 5′, 6′-CH₃ | 2′, 5′, 6′-CH₃ |
| 6′-CH₃ | 18.7, CH₃ | 1.42, d (6.1) | 6′ | 5′, 6′ | 5′, 6′ |

^{a}J values were determined in CD₂Cl₂ because the ¹H signals in MeOD were very broad
^{b}determined by HMBC correlations

TABLE 2
IC50 values of mayamycin against various test strains

| Test strain | IC50 [μM] |
|---|---|
| Bacillus subtilis (DSM 347) | 8.0 |
| Staphylococcus lentus (DSM 6672) | 8.0 |
| Staphylococcus aureus (ATCC 12600) | 2.5 |
| Staphylococcus aureus (ATCC 33593)* | 1.25 |
| Klebsiella pneumoniae (ATCC 700603)** | 2.5 |
| Staphylococcus epidermidis (Isolate***) | 0.31 |
| Pseudomonas aeruginosa (ATCC 10145)*** | 2.5 |
| Propionibacterium acnes (DSM 1897) | 31.2 |
| Xanthomonas campestris (DSM 2405) | 30.0 |
| Phytophthora infestans (Isolate) | 15.2 |

*methicillin-resistant
**extended β-lactamase resistance
***biofilm former

TABLE 3
IC50 values of mayamycin against tumor cell lines

| Cell line | | IC50 [μM] |
|---|---|---|
| HepG2 | liver tumor | 0.2 |
| HT29 | intestinal tumor | 0.3 |
| GXF251L | gastric tumor | 0.2 |
| LXF529L | lung tumor | 0.16 |
| MAXF401NL | breast cancer | 0.29 |
| MEXF426NL | melanoma | 0.13 |
| PAXF1657L | pancreatic tumor | 0.15 |
| RFX486L | kidney tumor | 0.33 |

1. A compound of the general structure 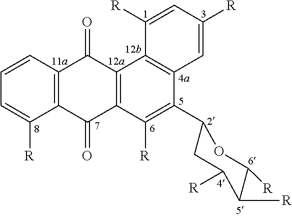
wherein
R is a hydrogen atom (H) or an unsubstituted, monosubstituted or polysubstituted C₁-C₂₀-alkyl, wherein the alkyl can be straight-chain, branched, cyclic or partially unsaturated, or an unsubstituted, monosubstituted or polysubstituted phenyl residue.

2. The compound of claim 1, wherein the substituents of the alkyl or phenyl residue are selected from the group consisting of a linear or branched alkyl residue, an acyl group, a halogen residue, an unsubstituted or alkyl-substituted amino group, a hydroxyl group, an ether group and a carboxyl group which is free, esterified with an alkyl group or amidated.

3. The compound of claim 2, wherein the acyl group is a formyl, acetyl, trichloroacetyl, fumaryl, maleyl, succinyl, benzoyl or a branched or hetero-atom- or aryl-substituted acyl group.

4. The compound of claim 2, wherein the compound is 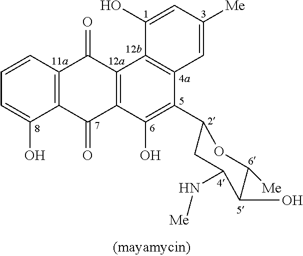 [SMILES: CNC1CC(c2c(O)c3c(c4c(O)cc(C)cc24)C(=O)c2cccc(O)c2C3=O)OC(C)C1O]
including diastereomers thereof.

5. A process for producing a compound defined by claim 1 comprising:
(a) culturing a bacterium of the genus Streptomyces; and
(b) isolating the compound from the culture medium and/or the bacterium.

6. The process of claim 5, wherein the bacterium is Streptomyces sp. HB202.

7. (canceled)

8. The method of claim 12, wherein the proliferative disease or disorder is selected from the group consisting of liver tumor diseases, intestinal tumor diseases, gastric tumor diseases, lung tumor diseases, breast cancer diseases, melanoma diseases, pancreatic tumor diseases and kidney tumor diseases.

9. (canceled)

10. (canceled)

11. A cosmetic product comprising a compound defined by claim 1.

12. A method of treating or preventing a proliferative disease or disorder in a patient comprising administering to the patient a compound defined by claim 1.

13. A method of treating or preventing an infectious disease, comprising administering a compound defined by claim 1.

14. The method of claim 13, wherein the infectious disease is a bacterial infection.

15. A method of treating or preventing a plant disease comprising administering a compound defined by claim 1.
